(19) United States
(12) Patent Application Publication
(10) Pub. No.: US 2005/0222679 A1
(43) Pub. Date: Oct. 6, 2005

(54) Bifocal implant and method for altering the refractive properties of the eye

(76) Inventor: Gholam A. Peyman, New Orleans, LA (US)

Correspondence Address: Bell, Boyd & Lloyd LLC, P.O. Box 1135, Chicago, IL, 60690-1135, US

(21) Appl. No.: 11/108,042

(22) Filed: Apr. 15, 2005

Related U.S. Application Data
(63) Continuation-in-part of application No. 10/784,169, filed on Feb. 24, 2004.
Application No. 10/784,169 is a continuation-in-part of application No. 10/406,558, filed on Apr. 4, 2003.
Application No. 10/784,169 is a continuation-in-part of application No. 10/356,730, filed on Feb. 3, 2003.
Application No. 10/356,730 is a continuation-in-part of application No. 09/843,141, filed on Apr. 27, 2001, now Pat. No. 6,551,307.
(60) Provisional application No. 60/449,617, filed on Feb. 26, 2003.

(51) Int. Cl. A61F 2/14

(52) U.S. Cl. 623/5.11; 623/5.12

(57) ABSTRACT

The present invention relates to an implant for insertion between layers of the cornea. The Implant includes a first portion adapted to change the curvature of the cornea at first area and thereby alter the refractive properties of the cornea at the first area, and a second portion adapted to change the refractive properties of a second area of the eye adjacent the first area and compensate for error at the second area caused by the first area change in curvature.

### CROSS-REFERENCE TO RELATED APPLICATION

This application is a continuation-in-part of U.S. patent application Ser. No. 10/784,169, filed Feb. 24, 2004, which is a continuation-in-part of U.S. patent application Ser. No. 10/406,558, filed Apr. 4, 2003, which claims the benefit of U.S. Provisional Application Serial No. 60/449,617, filed Feb. 26, 2003 and is a continuation-in-part of U.S. application Ser. No. 10/356,730, filed Feb. 3, 2003, which is a continuation-in-part of U.S. patent application Ser. No. 09/843,141, filed Apr. 27, 2001, now U.S. Pat. No. 6,551,307. The entire contents of each of which are herein incorporated by reference.

This application is related to U.S. application Ser. No. ______, entitled IMPLANT AND METHOD FOR ALTERING THE REFRACTIVE PROPERTIES OF THE EYE, filed Apr. 15, 2005, the entire contents of which are herein incorporated by reference.

### FIELD OF THE INVENTION

The present invention relates to a system and method for modifying the refractive properties in the live cornea of an eye. More particularly, the present invention relates to a system and method for modifying the live cornea by inserting an implant between corneal surfaces. The implant preferably has a first portion adapted to alter the refractive properties of a predetermined first area of the eye by displacing the first surface of the cornea at the first predetermined area, thereby changing the curvature of the cornea at the first predetermined area, and a second portion adapted to alter the refractive properties of a predetermined second area of the eye adjacent the first predetermined area, the second portion further adapted to compensate for error at the predetermined second area caused by the first portion.

### BACKGROUND

A conventional method for correcting the refractive error in a cornea is known as keratophakia, which involves implantation of a lens inside the cornea. Keratophakia uses an implant, which is placed into the cornea approximately equidistant from the exterior surface of the cornea and the interior surface. The procedure is usually done by first preparing a lens from corneal donor tissue or from synthetic material using a cryo-lathe. The lens is implanted by removing a portion of the cornea with a device called a microkeratomes, and the tissue is sutured back into place over the lens. However, there can be problems when microkeratomies are used for cutting the cornea. First, irregular keratectomies or perforations of the eye can result. Second, the recovery of vision can be rather prolonged.

Another surgical technique exists that uses a femtosecond laser to separate layers inside the stromal at least two-thirds of the distance from the top surface of the cornea to the inside of the eye. An incision is made to access this area, and a solid inlay is inserted to help correct myopia in the eye. However, separating the layers in the bottom two-thirds of the stroma makes it difficult to access the separated area to insert the inlay, and virtually impossible to change or modify the inlay without another extensive surgical procedure. This procedure also requires making an incision, which is parallel to the visual axis and is limited in the lateral direction by a maximum size of 0.3 mm to encase a relatively rigid inlay that forces the tissue in the lateral direction.

A further surgical technique exists that forms a flap-like portion of the live cornea, which is removed to expose an inner surface of the cornea. A blank is positioned on the exposed inner surface of the cornea, and a laser beam is then directed onto certain portions of the blank based on the type of ametropic condition (i.e., myopia, hyperopia or astigmatism) of the eye, so that the laser beam ablates those portions and thus reshapes the blank. The laser beam can also be directed onto certain portions of the exposed surface of the cornea to ablate those surfaces of the cornea. The flap-like portion of the cornea is repositioned over the remaining portion of the blank, so that the remaining portion of the blank influences the shape of the reattached flap-like portion of the cornea and thus modifies the curvature of the cornea. A more detailed description of this procedure is described in U.S. Pat. No. 5,919,185 to Peyman, the content of which is herein incorporated by reference.

Although this technique is very successful, this type of procedure may require ablation of a large portion of the blank, which results in the dispersion of a relatively large amount of heat. This heat can cause the lens to shrink and thus possibly inadvertently alter the intended refractive properties of the cornea, in which event correction will be less than desired or even irregular.

Additional surgical techniques exist that use ultraviolet light and short wavelength lasers to modify the shape of the cornea. For example, excimer lasers, such as those described in U.S. Pat. No. 4,840,175 to Peyman, the entire content of which is incorporated by reference herein, emit pulsed ultraviolet radiation that can be used to decompose or photoablate tissue in the live cornea to reshape the cornea. This technique is commonly known as the laser surgical technique known as laser in situ keratomycosis (LASIK).

In the LASIK technique, a portion of the front of the live cornea can be cut away in the form of a flap having a thickness of about 160 microns. This cut portion is removed from the live cornea to expose an inner surface of the cornea. A laser beam is then directed onto the exposed inner surface to ablate a desired amount of the inner surface up to 150-180 microns deep. The cut portion is reattached over the ablated portion of the cornea and assumes a shape substantially conforming to that of the ablated portion.

However, because only certain amount of cornea can be ablated without the remaining cornea becoming unstable or experiencing outbulging (eklasisa), this technique is not especially effective in correcting very high myopia. That is, a typical cornea is on average about 500 microns thick. The laser ablation technique requires that at least about 250 microns of the corneal stroma remain after the ablation is completed so that instability and outbulging do not occur.

Additional methods for correcting the refractive error in the eye include inserting an implant in-between layers of the cornea. Generally, this is achieved using several different methods. One method involves inserting a ring between layers of the cornea, as described in U.S. Pat. No. 5,405,384 to Silvestrini. Typically, a dissector is inserted in the cornea and forms a channel therein. Once it is removed, a ring is then inserted into the channel to alter the curvature of the cornea. In another method, a flap can be created similarly to the LASIK procedure and a lens can be inserted under the flap, as described in U.S. Pat. No. 5,722,971 to Peyman. A further method involves forming a pocket using an instrument, and inserting an implant into the pocket, as described in U.S. Pat. No. 4,655,774 to Choyce. The entire contents of each of these three patents are incorporated herein by reference.

However, with the above-described techniques, a knife or other mechanical instrument is generally used to form the channel, flap or pocket. Use of these instruments may result in damage or imprecision in the cut or formation of the desired area in which the implant is placed.

Therefore, there exists a need for an improved method of correcting refractive error in the cornea of an eye.

### SUMMARY

In one embodiment, a method of altering the refractive properties in an eye is provided. The method includes the step of separating layers of the cornea to form a first corneal layer and a second corneal layer, the first corneal layer facing in a posterior direction and the second corneal layer facing in an anterior direction. Next an implant is inserted between the first corneal layer and the second corneal layer, the implant including a first portion and a second portion, the first portion adapted to alter the refractive properties of the eye and the second portion adapted to correct error, including error caused by the first portion.

In another embodiment, an implant adapted to be positioned between a first surface of the cornea and a second surface of the cornea for altering refractive properties of an eye is provided. The implant includes a first portion adapted to alter the refractive properties of a predetermined first area of the eye by displacing the first surface of the cornea at the first predetermined area, thereby changing the curvature of the cornea at the first predetermined area, and a second portion adapted to alter the refractive properties of a predetermined second area of the eye adjacent the first predetermined area, the second portion further adapted to compensate for error at the predetermined second area caused by the first portion.

In another embodiment, an implant for insertion between layers of the cornea is provided. The implant includes a first portion adapted to change the curvature of the cornea at first area and thereby alter the refractive properties of the cornea at the first area, and a second portion adapted to change the refractive properties of a second area of the eye adjacent the first area and compensate for error at the second area caused by the first area change in curvature.

Other objects, advantages, and novel features of the present invention will become apparent to those skilled in the art from the following detailed description, which, taken in conjunction with the annexed drawings, discloses preferred embodiments of the invention.

### BRIEF DESCRIPTION OF THE DRAWINGS

Referring to the drawings which form a part of this disclosure:

FIG. 1 is a side view in section taken along the center of a myopic eye, showing the cornea, pupil and lens;

FIG. 2 is a side view in section of the eye of FIG. 1 with a flap formed in the surface of the cornea;

FIG. 3 is a top view of an implant according tone embodiment of the present invention;

FIG. 4 is a cross-sectional view of the implant of FIG. 3 taken along lines 4-4;

FIG. 5 is a side view in section of the eye of FIG. 2 with the implant of FIG. 3 positioned on an exposed surface of the cornea;

FIG. 6 is a side view in section of FIG. 5 with a laser ablating a portion of the implant;

FIG. 7 is a side view in section of the eye and implant of FIG. 5 with the flap repositioned over the implant;

FIG. 8 is a top view of a implant according to another embodiment of the present invention;

FIG. 9 is a cross-sectional view of the implant of FIG. 8 taken along lines 9-9;

FIG. 10 is a side view in section of the eye of FIG. 2 with the implant of FIG. 8 positioned on an exposed surface of the cornea; and

FIG. 11 is a side view in section of the eye and implant of FIG. 10 with the flap repositioned over the implant.

### DETAILED DESCRIPTION

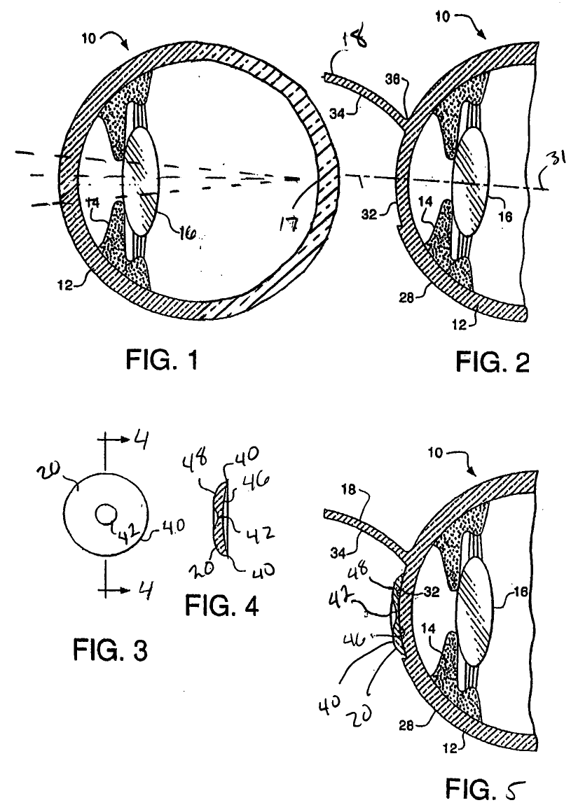

FIG. 1 is a side view in section taken through the center of an eye 10, which includes a cornea 12, a pupil 14 and a lens 16. As shown, the cornea 12 and lens 16 do not cooperatively focus light correctly on the retina 17 of the eye to provide adequate vision. More specifically, light passing through the lens 16 of eye 10 is focused in front of the retina. To correct this refractive error, the curvature of the cornea can be modified to correct the refractive power of the cornea and thus correct the manner in which the light is focused with respect to the retina. It is noted that although a myopic eye is shown, each of the embodiments described herein is not limited to correction of this specific refractive error, and the present devices and methods can be used to correct any suitable error (e.g. myopia, hyperopia, presbyopia, astigmatism, any suitable combination thereof and any other focusing errors in the eye or combinations thereof).

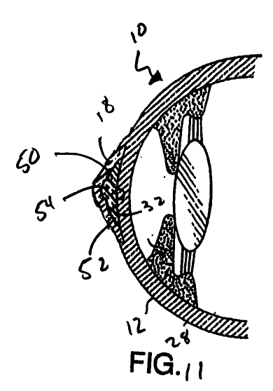

As seen in FIGS. 1-8, the refractive properties of eye 10 can be altered by forming a flap 18 in the cornea 12 and then placing inlay, lens or implant 20 under flap 18. Implant 20 is positioned adjacent the exposed surface of the cornea and can be easily shaped or ablated using a laser 24 (FIG. 6), if desired. It is not necessary to ablate the implant 20, if the eye is properly corrected without ablation. Furthermore, the ablation can take place immediately upon placement on the surface of the cornea, or at any later time.

To begin, the refractive error in the eye is measured using wavefront technology, as is known to one of ordinary skill in the art. The refractive error measurements are used to determine the appropriate lens or implant 20 to best correct the error in the patient's cornea. Preferably, the lens 20 is manufactured or shaped prior to the use of the wavefront technology and is stored in a sterilized manner until that specific lens shape or size is needed, or stored in any suitable manner. However, the information received during the measurements from the wavefront technology can be used to form the lens using a cryo-lathe, laser, or any other desired system, machine or device.

A flap or portion 18 can be formed in the surface 28 of the cornea 12, as seen in FIG. 2. The flap may be formed be any means desired, such as with a knife, microkertome, or with a laser. Preferably an internal area of the cornea is separated into first 32 and second 34 substantially circular shaped internal surfaces to form the circular shaped corneal flap 18. First internal surface 32 faces in an anterior direction of cornea 12 and the second internal surface 34 faces in a posterior direction of the cornea 12. The flap 18 preferably has a uniform thickness of about 10-250 microns, and more preferably about 80-100 microns, but can be any suitable thickness. That is, the flap can be formed such that it separate layers of the stroma or layers of the epithelium, separates the epithelium from the Bowman's Layer, or separates the Bowman's layer from the stroma, or the flap can be formed in any other portion or suitable layer of the cornea.

A portion 36 of flap 18 preferably remains attached to the cornea by an area at the periphery 30 of the flap. However, the flap can be any suitable configuration, such as a flap attached to the cornea at a location other than at the periphery or a flap that is not attached to the cornea at all. Additionally, the flap may be shaped or sized as desired and does not need to be circular.

The flap is removed or moved or rotated about portion 36 using any suitable device or any device known in the art, such as spatula or microforceps or any other device, to expose the first and second corneal surfaces 32 and 34, respectively. The flap preferably exposes a portion of the corneal surface 32 that intersects the main optical axis 31 and allows uninhibited access thereto.

Implant 20 can then be positioned adjacent the surface 32 of the cornea. Implant 20 is preferably any polymer or hydrogel having about 50% water content; however, the water content can be any percentage desired. The implant may be formed from synthetic or organic material or a combination thereof. For example, the implant can be collagen combined with or without cells; a mixture of synthetic material and corneal stromal cells; silicone or silicone mixed with collagen; methylmetacrylate; any transparent material, such as polyprolidine, polyvinylpylidine, polyethylenoxyde, etc.; or any deformable polymer, which can change its shape with radiation after implantation.

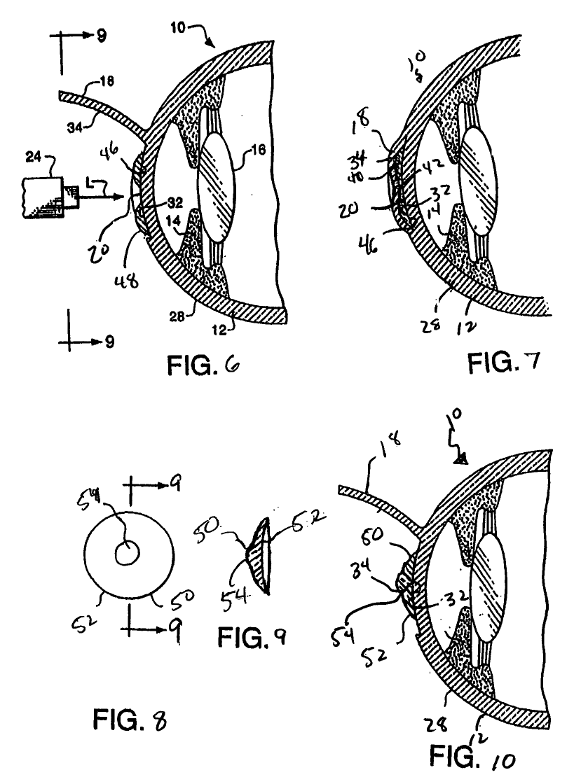

As shown in FIGS. 3 and 4, implant 20 is preferably substantially circular and has a first surface 46 and a second surface 48. First surface 46 preferably is positioned adjacent a surface of the cornea and second surface 48 preferably is opposite the first surface. Implant 20 preferably has a first substantially ring-shaped or peripheral portion 40 and a second substantially circular or central portion 42. The diameter of the central portion 42 is preferably between about 1.5 mm and 2.5 mm and the diameter of the implant overall is preferable between about 1.5 mm and 6 mm. Additionally, implant 20 can be porous to allow oxygen and nutrients to pass therethrough. The implant can have any suitable thickness and is generally between about 5-2000 microns, and more preferably less than 200 microns. The inside edge can be thinner or thicker than the outside edge; for example, the inside edge can have a thickness of about 1-100 microns, while the outside edge has a thickness of about 20-3000 microns. However, it is noted that the implant and the first and second portions can have any desired thickness or configuration that would allow it to elevate or move any portion of surface 34 relative to surface 32. The thickness and position of implant 20 generally defines the degree of correction of the cornea. Furthermore, the implant 20 is not limited to a bifocal lens and can have multiforal properties. That is the implant 20 can have as many or as few portions as desired, and the portions can be locate in any area of the implant

Preferably, implant 20 is formed from an ablatable polymer and has at least one and more preferably several hundred physical openings or microperforations formed as passageways from the first surface of the implany through the inlay to the second surface of the inlay. Each microperforation is about 0.1 microns to about 500 micros in diameter and extends from the first surface of the implant to the second surface. These perforations form a net in the implant, and permit molecules of oxygen, water, solute and protein to permeate through the implant with substantially no or no inhibition. Any or all of the microperforations or openings in- the any of the implants described herein can have a glare-free material disposed thereon, if desired. For a further discussion of glare-free material, refer to U.S. Pat. Nos. 6,280,471 and 6,277,146 both to Peyman et al., the entire contents of both of which are incorporated herein by reference. It is noted that is not necessary to have either the perforations or the glare-free material describe herein.

Additionally, implant 20 can have substantially the same refractive index as the cornea or any other suitable index. For example, the implant 20 can have a index of refraction that is substantially higher than that of the cornea (i.e., up to about 1.76). Examples of suitable materials have been developed Nitto Denko Corporation and Brewer Science. Nitto Denko has increased the index of refraction of thermosetting resin by the addition of titania, zirconia and other metal oxide nanoparticles or the additional of titanium dioxide, zirconium dioxide and other metal oxide materials. Brewer Science has also developed a new approach to the preparation of hybrid coating systems where the high index metal oxide component forms spontaneously during the curing process of the coating, leaving the polymer and metal oxide phases at a near molecular-level of interdispersion. The resulting coatings have refractive indices ranging from 1.6 to as high as 1.9 (in the range of 400 to 700 nm) depending on the metal oxide loading. This high refractive index allows the lens to be thinner than a conventional lens, and still alter the refractive characteristics of the cornea. This high refractive material allows the lens to be thinner than a conventional lens, and still alter the refractive characteristics of the cornea. If formed from this material, the lens can have a thickness of between about 0.5 microns and 30 microns. Such a thickness allows the refractive properties of the eye to be altered using the refractive index of the lens and/or changing the curvature of the surface of the cornea.

The central portion 42 preferably has a different refractive power and/or index than the peripheral portion and each are configured or adapted to correct a different error in the eye or change the curvature of a specific area or portion of the cornea.

For example the implant shown in FIGS. 3-7 is configured to correct myopia and presbyopia. Preferably the central portion corrects presbyopia and the peripheral portion corrects myopia, thus forming a bifocal lens. Further, the central portion is configured to correct error or curvature change in the central portion of the eye that is caused or is a result of the curvature change effected by the peripheral portion. In other words, when correcting the myopic condition in the eye by altering the curvature of the cornea overlying the peripheral area of the implant 20, the overall curvature of the cornea is flattened, this flattening of the cornea effects the central portion and any correction thereof must compensate of the peripheral curvature change.

The following is a chart showing the dipoter power changes necessary to correct a myopic and presbyotic eye:

| | Perpheral Correction | Central Correction |
|---|---|---|
| Myopia (Diopters) | (Diopters) | (Diopters) |
| −5.00 | −5.00 | −2.00 |
| −4.00 | −4.00 | −1.00 |
| −3.00 | −3.00 | +0.50 |
| −2.00 | −2.00 | +1.00 to +1.50 |
| −1.00 | −1.00 | +2.00 to +2.50 |

It is noted that the peripheral measurements or corrective powers and the central measurements or corrective powers can be switched, that is the central portion can be used to correct myopia and the peripheral portion can be used to correct presbyopia.

FIGS. 9-11 illustrate an implant 50 according to a second embodiment of the present invention. Implant 50 is substantially similar to implant 20 and any description thereof is applicable to implant 50, unless specifically described herein. Implant 50 has a first or peripheral portion 52 and a second or central portion 54. Each portion is preferably configured to correct a specific error at a predetermined portion or area in the eye, creating bifocal vision correction.

The central portion preferably has a different refractive power and/or index than the peripheral portion and each are configured or adapted to correct a different error in the eye or change the curvature of a specific area or portion of the cornea.

For example the implant shown in FIGS. 9-11 is configured to correct hyperopia and presbyopia. Preferably the central portion corrects presbyopia and the peripheral portion corrects hyperopia, thus forming a bifocal lens. Further, the central portion is configured to correct error or curvature change in the central portion of the eye that is caused or is a result of the curvature change effected by the peripheral portion. In other words, when correcting the hyperopic condition in the eye by altering the curvature of the cornea overlying the peripheral area of the implant 50, the overall curvature of the cornea is steepened. This steepening of the cornea effects the central portion and any correction thereof must compensate of the peripheral curvature change.

The following is a chart of the preferred power correction for each portion of the implant 50:

| | Peripheral Portion | Central Portion |
|---|---|---|
| Hyperopia (Diopters) | (Diopters) | (Diopters) |
| +5.00 | +5.00 | +8.00 |
| +4.00 | +4.00 | +7.00 |
| +3.00 | +3.00 | +6.00 |
| +2.00 | +2.00 | +5.00 |
| +1.00 | +1.00 | +4.00 |

It is noted that the peripheral measurements or corrective powers and the central measurements or corrective powers can switched, that is the central portion can be used to correct hyperopia and the peripheral portion can be used to correct presbyopia

Furthermore, the method and implants described herein are suitable for the correction of prespyopia in an emmetropic eye. As with the above described embodiments to correct the vision in an eye having this type of vision problems, it is beneficial to produce a bifocal implant. For example, the type of implant shown in FIGS. 3 and 4 would be suitable for this type of correction. The implant is preferably configured such that the peripheral portion corrects presbyopia and the central portion corrects error in the cornea at the area overlying the central portion caused by the lifting of the cornea in the area overlying the peripheral portion. This correction is generally required, since as described above, the plus diopter correction or the lifting of cornea for the presbyopic correction causes an overall steepening of the cornea. Central portion of the cornea generally needs to be flattened to compensate for the peripheral presbyotic correction. For example, in a presbyoptic eye that requires a +3.00 diopter correction in the peripheral portion of the implant, −0.1 to −0.5 diopter correction must be effected in the central portion of the implant.

It is noted that the implants described herein can be configured to alter the refractive properties in any manner desired. For example, any of the herein described implants can be configured to correct hyperopia, myopia, astigmatism, and/or presybyopia any combination thereof, or any known refractive error or combination of known refractive errors.

Formation of the Implant

Each of the herein described embodiments can be formed in a substantially similar manner using a device to calculate the required diopter power (such as a computer, the tables described herein or any other device or method) and a cyro-lathe, laser of other device for forming the implant.

Preferably, the implant is formed as a multifocal implant that is adapted or configured for insertion between layers of a cornea of an eye, such as under the above described flap; however, it is noted that the implant can be inserted between layers of the cornea in any desired manner. For example, the implant can be inserted into a pocket or opening in the cornea. As described above, the implant is configured to change the refractive properties of the eye by changing the curvature of the cornea.

First, a specific eye or a general known problem of an eye or eyes is determined. For example, a specific eye can have a myopic, hyperopic, astigmatic or presbyotic condition. The specific refractive error of this condition in the eye is determined. Additionally, it is within the scope of the present invention that a known condition (based on knowledge in the field or a model) in an eye can be determined.

The appropriate correction is determined and a first portion, such as the periphery, of the implant is formed such that the first portion is configured to alter the corneal curvature at a first predetermined area of the eye and thus correct the refractive error at the first predetermined area.

Then a determination of the refractive error caused by the first portion altering the corneal curvature at a second predetermined area of the eye is made. The eye can have a second refractive error at this point in addition to the error caused by the first portion. For example, if the first refractive error in the eye is myopia, then second refractive error could be presbyopia. If this is the case, an overall correction could be made at this point, i.e., the presbiotic correction plus the correction required to compensate for the first portion.

A second portion of the implant is then formed such that the second portion of the implant is configured to alter the corneal curvature at the second predetermined area and thus correct the refractive error caused by the first portion and/or the second refractive error.

It is noted that the above examples are merely to facilitate understanding of the present invention and are in no way meant to limit the present invention to the above described combination of refractive error. In other words, any know suitable combination of refractive errors can be corrected by the above described implants.

It is noted that in each of the above described embodiments, the specific portions are not limited to the peripheral portion and/or the central portion. That the correction of myopia, hyperopia, astigmatism and/or presbyopia can be in configured to be in any portion or area of the implant, and the bifocal properties can be concentric, eccentric asymmetric, symmetric or in any suitable position or configuration desired.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

1. A method of altering the refractive properties in an eye, comprising the steps of
separating layers of the cornea to form a first corneal layer and a second corneal layer, the first corneal layer facing in a posterior direction and the second corneal layer facing in an anterior direction,
inserting an implant between the first corneal layer and the second corneal layer, said implant including a first portion and a second portion, said first portion adapted to alter the refractive properties of the eye and the said second portion adapted to correct error, including error caused by said first portion.

2. A method according to claim 1, wherein
said first portion is at the periphery of the implant.

3. A method according to claim 2, wherein
said second portion as at the center of the implant and is substantially surrounded by said first portion.

4. A method according to claim 1, wherein
said first portion is configured to correct presbyopia and said second portion is configured to correct at least one of myopia and hyperopia.

5. A method according to claim 1, wherein
said second portion is configured to correct presbyopia and said first portion is configured to correct at least one of myopia and hyperopia.

6. A method according to claim 1, wherein
said step of separating layers of the cornea includes separating layers of the cornea to form a flap attached to the cornea at a peripheral portion thereof.

7. An implant adapted to be positioned between a first surface of the cornea and a second surface of the cornea for altering refractive properties of an eye, comprising:
a first portion adapted to alter the refractive properties of a predetermined first area of the eye by displacing the first surface of the cornea at said first predetermined area, thereby changing the curvature of the cornea at said first predetermined area; and
a second portion adapted to alter the refractive properties of a predetermined second area of the eye adjacent said first predetermined area, said second portion further adapted to compensate for error at said predetermined second area caused by said first portion.

8. A method according to claim 7, wherein said first portion is at the periphery of the implant.

9. A method according to claim 8, wherein
said second portion as at the center of the implant and is substantially surrounded by said first portion.

10. A method according to claim 9, wherein
said first portion is configured to correct presbyopia and said second portion is configured to correct at least one of myopia and hyperopia.

11. A method according to claim 9, wherein
said second portion is configured to correct presbyopia and said first portion is configured to correct at least one of myopia and hyperopia.

12. An implant for insertion between layers of the cornea, comprising:
a first portion adapted to change the curvature of the cornea at first area and thereby alter the refractive properties of the cornea at said first area; and
a second portion adapted to change the refractive properties of a second area of the eye adjacent the first area and compensate for error at said second area caused by said first area change in curvature.

13. An implant according to claim 12, wherein
said implant is substantially circular and said first portion is substantially ring-shaped and at the periphery of said implant.

14. An implant according to claim 12, wherein
said second portion is substantially at the center of said implant and surrounded by said first portion.

15. An implant according to claim 14, wherein
said second portion is configured to correct presbyopia.

16. An implant according to claim 15, wherein
said first portion is configured to correct at least one of myopia and hyperopia

17. An implant according to claim 14, wherein
said first portion is configured to presbyopia.

18. An implant according to claim 17, wherein
said second portion is configured to correct at least one of myopia and hyperopia.

19. An implant according to claim 17, wherein
said second portion is configured to return the refractive properties of the second area to its original emmetropic condition.

20. An implant according to claim 14, wherein
said implant is sized and configured to be substantially centered about the main optical axis of the eye.

21. A method of forming a multifocal implant for insertion between layers of a cornea of an eye, the implant configured to change the refractive properties of the eye, the method comprising the steps of
determining a first refractive error of a first condition in the eye,
forming a first portion of the implant such that the first portion is configured to alter the corneal curvature at a first predetermined area of the eye and thus correct the first refractive error at the first predetermined area,
determining a refractive error caused by the first portion altering the corneal curvature at a second predetermined area of the eye, and
forming a second portion of the implant such that the second portion of the implant is configured to alter the corneal curvature at the second predetermined area and thus correct the refractive error caused by the first portion.

22. A method of forming a multifocal implant according to claim 21, further comprising the steps of
determining a second refractive error of a second condition in the eye, and
forming the second portion of the implant such that the implant is configured to alter the corneal curvature of the second predetermined area to thus correct a combined refractive error caused by the first portion and the second condition.
